(19) United States
(12) Patent Application Publication
(10) Pub. No.: US 2025/0241860 A1
(43) Pub. Date: Jul. 31, 2025

(54) HOLLOW MULTILAYER METAL-ORGANIC-FRAMEWORK MATERIAL AND PREPARATION METHOD AND APPLICATION THEREOF

(71) Applicants: Zhejiang University of Technology, Hangzhou (CN); Zhejiang Provincial Hospital of Traditional Chinese Medicine, the First Affilliated Hospital, Hangzhou (CN)

(72) Inventors: Juan Jin, Hangzhou (CN); Wei Shao, Hangzhou (CN); Jingjing Li, Hangzhou (CN); Qiang He, Hangzhou (CN)

(21) Appl. No.: 18/661,735

(22) Filed: May 13, 2024

(30) Foreign Application Priority Data
Jan. 25, 2024 (CN) 202410104626.0

(51) Int. Cl. A61K 9/48; A61K 31/37; A61K 38/48; A61P 7/02

(52) U.S. Cl. CPC A61K 9/4816; A61K 31/37; A61K 38/48; A61P 7/02

(57) ABSTRACT

A hollow multilayer MOF material and its preparation method and application relates to the field of nano drug delivery therapy technology. A ZIF-67@ZIF-8@ZIF-67@ZIF-8 hollow multilayer MOF material is applied as a thrombus drug nanocarrier to prepare drugs for thrombus treatment. In vitro experiments indicate that the thrombus drug nanocarrier has an efficient targeting effect and an excellent sustained release effect in combination with streptokinase and warfarin, and the thrombus drug nanocarrier has a good therapeutic effect and the long-term anticoagulant effect.

### TECHNICAL FIELD

The disclosure relates to the field of nano drug delivery therapy technology, and more particularly to a hollow multilayer metal-organic-framework (MOF) material and its preparation method and application.

### BACKGROUND

Venous thrombosis refers to the formation of thrombi and other blockages in veins, one of common clinical manifestations is deep vein thrombosis, when an embolism is dislodged from blood vessels and circulates with blood, if the embolism reaches lungs and blocks pulmonary arteries, pulmonary embolism can be caused, with a fatality rate as high as in a range of 9%-50%. The venous thrombosis often occurs in legs and is more common in elderly, obese people, people who have undergone major surgery, and people who have suffered leg injuries, etc. A global incidence of the venous thrombosis in the general population is in a range of 1/1000-3/1000, and an incidence of the venous thrombosis in China is also very high. Patients with the venous thrombosis do not show apparent symptoms in an early stage, often with slight swelling, redness, fever and other symptoms that can be easily ignored, once disease onset occurs, in addition to life-threatening, it often leaves sequelae such as disability etc., which seriously affects life quality.

At present, a treatment of the venous thrombosis mainly includes two steps. A first step is thrombolysis, i.e., some thrombolytic drugs are used to dissolve thrombi. A second step is anticoagulation, the thrombi are highly prone to reform at sites of illness, and anticoagulant drugs can be used to inhibit a recurrence of the thrombi. However, the current treatment method has a series of drawbacks that urgently need to be addressed: 1. The thrombolytic drugs have a relatively short residence time in vivo, and thus in order to achieve good therapeutic effects, high doses of the thrombolytic drugs are usually required. At the same time, the thrombolytic drugs may also be accompanied by side effects such as major bleeding and unstable blood pressure, especially for high-risk groups such as pregnant women or the elderly; 2. A drug utilization rate is low, often less than 5% of drugs may reach the site of the thrombi to complete treatment; 3. Drugs can only stay on a surface of the thrombus and cannot effectively penetrate into the thrombus, which greatly reduces therapeutic effects to the thrombi.

### SUMMARY

The disclosure aims at providing a hollow multilayer MOF material and its preparation method and application to solve above problems in the related art.

To achieve above purposes, the disclosure provides following technical solutions.

The disclosure provides a preparation method of a zeolitic-imidazolate-framework-67 (ZIF-67)@zeolitic-imidazolate-framework-8 (ZIF-8)@ZIF-67@ZIF-8 hollow multilayer MOF material, including following steps.

(1) First cobalt nitrate methanol solution is mixed with first 2-methylimidazole methanol solution to undergo a reaction to obtain a first sample.

(2) First zinc nitrate methanol solution and second 2-methylimidazole methanol a solution are added to the first sample to undergo a reaction to obtain a second sample.

(3) Second cobalt nitrate methanol solution and third 2-methylimidazole methanol solution are added to the second sample to undergo a reaction to obtain a third sample.

(4) Second zinc nitrate methanol solution and fourth 2-methylimidazole methanol solution are added to the third sample to undergo a reaction, followed by centrifuging to obtain precipitate.

(5) The precipitate in the step (4) is dispersed in water, aqueous solution of ethylene glycol and aqueous solution of triethanolamine are added to the water added with the precipitate to obtain a mixture, and the mixture is stirred to obtain the ZIF-67@ZIF-8@ZIF-67@ZIF-8 hollow multilayer MOF material.

In an embodiment, conditions of the reaction in each of the step (1) to the step (4) include: stirring at a temperature in a range of 50-60 degrees Celsius (° C.) with a speed in a range of 200-600 revolutions per minute (rpm) for 5 minutes (min), followed by standing for 1 hour (h).

In an embodiment, a concentration of each of the first cobalt nitrate methanol solution and the second cobalt nitrate methanol solution is in a range of 0.01-0.5 mole per liter (mol/L), a concentration of each of the first 2-methylimidazole methanol solution to the fourth 2-methylimidazole methanol solution is in a range of 1-5 mol/L, a concentration of each of the first zinc nitrate methanol solution and the second zinc nitrate methanol solution is in a range of 0.01-0.5 mol/L, and a volume ratio of the first cobalt nitrate methanol solution to the first 2-methylimidazole methanol solution is in a range of 1:1-20, a volume ratio of the first zinc nitrate methanol solution to the second 2-methylimidazole methanol solution is in a range of 1:1-20, a volume ratio of the second cobalt nitrate methanol solution to the third 2-methylimidazole methanol solution is in a range of 1:1-20, and a volume ratio of the second zinc nitrate methanol solution to the fourth 2-methylimidazole methanol solution is in a range of 1:1-20.

In an embodiment, the centrifuging includes: centrifuging the third sample added with the third product at a rotate speed in a range of 5000-10000 rpm for 15-30 min.

In an embodiment, the stirring includes: stirring the mixture at a temperature in a range of 0-15° C. with a stirring speed in a range of 500-1000 rpm for 2-6 h.

In an embodiment, a concentration of the aqueous solution of the ethylene glycol is in a range of 0.2-1.2 grams per milliliter (g/mL), a concentration of the aqueous solution of the triethanolamine is in a range of 0.02-0.5 g/mL, and a ratio of the precipitate to the aqueous solution of the ethylene glycol to the aqueous solution of the triethanolamine is in a range of 1 milligram (mg):(5-10 mL):(0.5-2 mL).

The disclosure further provides the ZIF-67@ZIF-8@ZIF-67@ZIF-8 hollow multilayer MOF material prepared through the preparation method above.

The disclosure further provides an application of the ZIF-67@ZIF-8@ZIF-67@ZIF-8 hollow multilayer MOF material as a thrombosis drug nanocarrier to prepare drugs for thrombosis treatment.

The disclosure further provides an antithrombotic pharmaceutical formulation, including the ZIF-67@ZIF-8@ZIF-67@ZIF-8 hollow multilayer MOF material and an active ingredient.

In an embodiment, the active ingredient includes at least one selected from the group consisting of streptokinase, urokinase, plasmin, low molecular weight heparin sodium, rivaroxaban, and warfarin.

Based on the above technical solutions, the disclosure has below beneficial effects.

The disclosure provides the ZIF-67@ZIF-8@ZIF-67@ZIF-8 hollow multilayer MOF material and its application as the thrombosis drug nanocarrier to prepare the drugs for the thrombosis treatment. In vitro experiments have shown that the thrombosis drug nanocarrier has an efficient targeting effect, and an excellent sustained release effect in combination with streptokinase and warfarin. The thrombosis drug nanocarrier has an excellent therapeutic and long-term anticoagulant effect on venous thrombosis.

### BRIEF DESCRIPTION OF DRAWINGS

Accompanying drawings are described below to provide a clearer description of technical solutions in embodiments of the disclosure or in the related art. It is apparent that the accompanying drawings described below are only some of the embodiments of the disclosure, and for those skilled in the art, other accompanying drawings can be obtained according to the accompanying drawings described below without creative labor.

FIG. 1 illustrates a photograph of a ZIF-67@ZIF-8@ZIF-67@ZIF-8 hollow multilayer MOF material under a transmission electron microscope.

FIG. 2 illustrates schematic targeted fluorescence imaging of a ZIFs antithrombotic pharmaceutical formulation on venous thrombosis in a leg;

FIG. 3A illustrates a release kinetics curve of a ZIF-67@ZIF-8@ZIF-67@ZIF-8 hollow multilayer MOF material for a thrombolytic drug, i.e., streptokinase;

FIG. 3B illustrates a release kinetics curve of a ZIF-67@ZIF-8@ZIF-67@ZIF-8 hollow multilayer MOF material for an anticoagulant drug, i.e., warfarin;

FIG. 4 illustrates photographs of an in vitro experiment of a ZIFs antithrombotic pharmaceutical formulation on thrombosis treatment at 0, 1, 2 and 3 h.

FIG. 5 illustrates photographs of an in vivo experiment in mice of a ZIFs antithrombotic pharmaceutical formulation on thrombosis treatment at 0 and 6 h.

### DETAILED DESCRIPTION OF EMBODIMENTS

Multiple embodiments of the disclosure are described below, the detailed description should not be considered as a limitation of the disclosure, but should be understood as a further detailed description of certain aspects, features and embodiments of the disclosure.

It should be noted that terms in the disclosure are only for describing the embodiments, and not intended to limit the disclosure. In addition, a numerical range in the disclosure should be understood as each intermediate value between an upper limit and a lower limit of the numerical range are disclosed. Each smaller range between any stated value or any intermediate value in a stated range, as well as any other stated value or any intermediate value in the stated range is also included in the disclosure. An upper limit and a lower limit of each smaller range can be included in or excluded from each smaller range independently.

Unless otherwise specified, all technical and scientific terms used in the disclosure have same meanings as commonly understood by those skilled in the art. Although only preferred methods and materials are described in the disclosure, any methods and materials similar or equivalent to those described in the disclosure may also be used in implementation or testing of the disclosure. All literature referred to in this specification is incorporated by reference for a purpose of disclosing and describing methods and/or materials associated with the literature. In an event of conflict with any incorporated literature, contents of the specification shall prevail.

It is apparent for those skilled in the art that various improvements and variations can be made to the embodiments in the specification of the disclosure without departing from the scope or sprit of the disclosure. It is apparent for those skilled that other embodiments may be obtained according to the specification of the disclosure. The embodiments and the specification of the disclosure are exemplary only.

Terms in the disclosure, such as “include”, “have” and “contain” etc., are all open-ended terms, i.e., meant to include but not be limited to.

Technical solutions described in the disclosure, if not specified, are conventional in the art, and reagents or raw materials used, if not specified, are purchased from commercial sources or are publicly available.

### Embodiment 1

1. At 55° C., 2 mL of cobalt nitrate methanol solution at 0.05 mol/L is mixed with 4 mL of 2-methylimidazole methanol solution at 0.02 mol/L and then stirred at a rotate speed in a range of 200-600 rpm for 5 min followed by standing for 1 h to obtain a sample 1.

2. At 55° C., 1 mL of zinc nitrate methanol solution at 0.05 mol/L and 2 mL of 2-methylimidazole methanol solution at 0.05 mol/L are added to the sample 1 and then stirred at a rotate speed in a range of 200-600 rpm for 5 min, followed by standing for 1 h to obtain a sample 2.

3. At 55° C., 1 mL of the cobalt nitrate methanol solution at 0.05 mol/L and 2 mL of the 2-methylimidazole methanol solution at 0.05 mol/L are added to the sample 2 and then stirred at a rotate speed in a range of 200-600 rpm for 5 min followed by standing for 1 h to obtain a sample 3.

4. At 55° C., 1 mL of the zinc nitrate methanol solution at 0.05 mol/L and 2 mL of the 2-methylimidazole methanol solution at 0.05 mol/L are added to the sample 3 and then stirred at a rotate speed in a range of 200-600 rpm for 5 min, followed by standing for 1 h to obtain a sample 4.

5. At 15° C., 5 mg of the sample 4 is taken and dispersed in 30 mL deionized water, and then added with 30 mL of 0.2 g/mL aqueous solution of ethylene glycol and 3 mL of 0.1 g/mL aqueous solution of triethanolamine to obtain a mixture, the mixture is stirred at 600 rpm for 4 h to obtain a sample 5, i.e., a ZIF-67@ZIF-8@ZIF-67@ZIF-8 hollow multilayer MOF material.

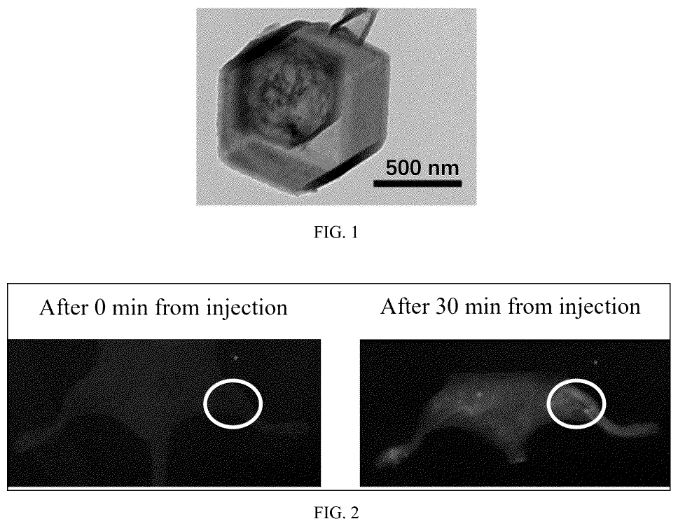

The ZIF-67@ZIF-8@ZIF-67@ZIF-8 hollow multilayer MOF material is characterized by a transmission electron microscope, as shown in FIG. 1, the ZIF-67@ZIF-8@ZIF-67@ZIF-8 hollow multilayer MOF material has multiple graded pores that can be loaded with various thrombolytic and anticoagulant drugs of different sizes.

### Embodiment 2

2 mg of the sample 5 is taken and added with 10 mL of 1 g/L streptokinase solution and 10 mL of 1 g/L warfarin solution and then stirred for 12 h, followed by being dispersed in 10 mL water to obtain a ZIF-67@ZIF-8@ZIF-67@ZIF-8 hollow multilayer MOF material loaded with thrombus treatment drugs, i.e., a ZIFs antithrombotic pharmaceutical formulation at a concentration of 0.2 g/L.

Hair is removed from a leg of an anesthetized mouse, then epidermis of venous blood vessels in the leg of the anesthetized mouse are scratched by a surgical knife, then skin near the venous blood vessels is cut, and a gauze containing ferric chloride aqueous solution at 1 mol/L is applied to an anatomical site of the leg of the anesthetized mouse for 30 min to obtain a mouse model with venous thrombosis in the leg.

50 microliters of the ZIFs antithrombotic pharmaceutical formulation at 0.2 g/L is injected intravenously to the mouse model with the venous thrombosis in the leg, after 30 min, fluorescence imaging of the venous thrombosis in the leg can be seen as shown in FIG. 2. FIG. 2 illustrates that after 30 min from the intravenous injection, there are apparent fluorescence signals on the venous thrombosis in the leg, indicating that the ZIFs antithrombotic pharmaceutical formulation has an efficient targeting effect.

### Embodiment 3

50 mL of the ZIFs antithrombotic pharmaceutical formulation at 0.2 g/L is taken and added with 25 mL of 1 mol/L streptokinase solution and 25 mL of 1 mol/L warfarin solution and then stirred for 24 h to obtain a ZIFs antithrombotic pharmaceutical formulation loaded with drugs. The ZIFs antithrombotic pharmaceutical formulation loaded with drugs is centrifuged, then washed, and then dispersed in 100 mL water to obtain a mixture, the mixture is stirred slowly at a rotate speed of 1000 rpm, samples are taken from the stirred mixture every 30 min to determine liquid chromatography to observe release of the streptokinase and other samples are taken from the stirred mixture every 10 days to determine the liquid chromatography to observe release of the warfarin.

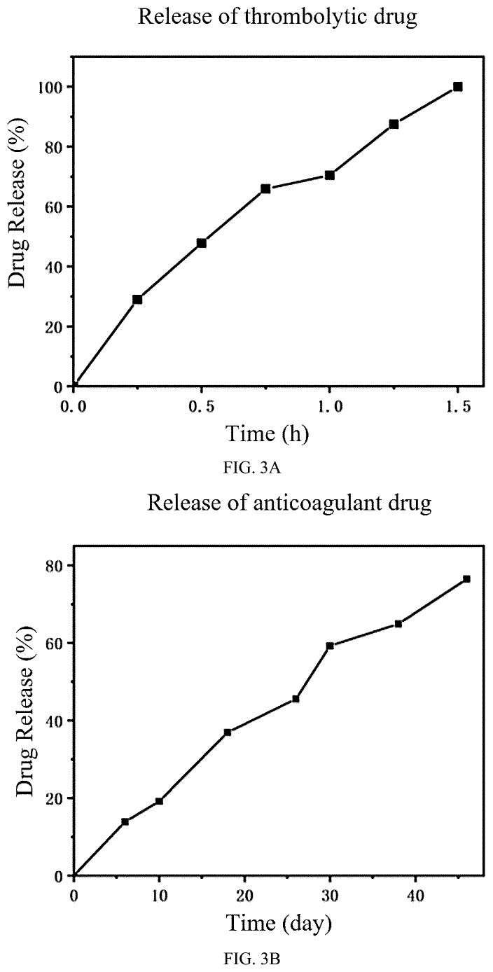

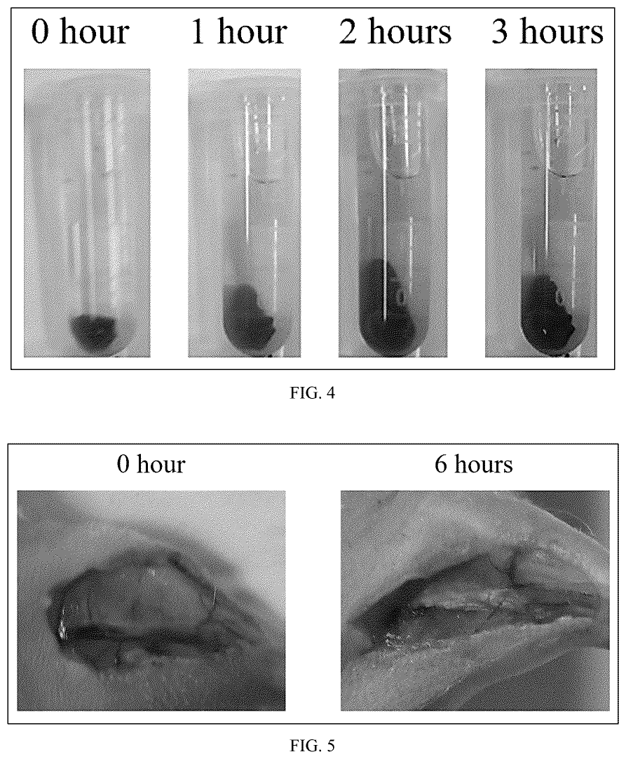

Results show that, for a large-sized thrombolytic drug, i.e., the streptokinase at a molecular weight of 47286.86 g/mol, drug release time of a nanocarrier material of the ZIFs antithrombotic pharmaceutical formulation loaded with drugs is 5 h, and for a small-sized anticoagulant drug, i.e., the warfarin at a molecular weight 308.33 g/mol, drug release time of the nanocarrier material of the ZIFs antithrombotic pharmaceutical formulation loaded with drugs is 45 days. It is indicated that the nanocarrier material provided in the disclosure has an excellent sustained release effect, as shown in FIG. 3A and FIG. 3B.

### Embodiment 4

0.5 mL of the ZIFs antithrombotic pharmaceutical formulation is injected intravenously to the mouse model with the venous thrombosis in the leg constructed in the embodiment 2, after 6 h, the venous thrombosis is observed to be disappeared at the naked eye, indicating that the ZIFs antithrombotic pharmaceutical formulation prepared in the disclosure has a rapid thrombolytic effect. In the next month, the site of the thrombosis venous is observed continuously and no thrombus reappeared, indicating that the ZIFs antithrombotic pharmaceutical formulation has a long-term anticoagulant effect.

In summary, the disclosure provides the ZIF-67@ZIF-8@ZIF-67@ZIF-8 hollow multilayer MOF material and its application as a thrombosis drug nanocarrier to prepare drugs for the thrombosis treatment. In vitro experiments proves that the thrombosis drug nanocarrier has the efficient targeting effect, and the excellent sustained release effect in combination with the streptokinase and the warfarin, and the thrombosis drug nanocarrier has a good therapeutic effect and the long-term anticoagulant effect.

It is apparent that above embodiments of the disclosure are only intended to illustrate the disclosure and are not intended to limit implementation method of the disclosure. For those skilled in the art, other variations or changes in different forms can be made on the basis of above description. It is neither necessary nor possible to exhaust all embodiments herein. Any modifications, equivalent substitutions and improvements made within the spirit and principles of the disclosure shall be included in the protection scope of the claims of the disclosure.

What is claimed is:

1. A preparation method of a zeolitic-imidazolate-framework-67 (ZIF-67)@zeolitic-imidazolate-framework-8 (ZIF-8)@ZIF-67@ZIF-8 hollow multilayer metal-organic-framework (MOF) material, comprising following steps:
(1) mixing first cobalt nitrate methanol solution with first 2-methylimidazole methanol solution to undergo a reaction to obtain a first sample;
(2) adding first zinc nitrate methanol solution and second 2-methylimidazole methanol solution to the first sample to undergo a reaction to obtain a second sample;
(3) adding second cobalt nitrate methanol solution and third 2-methylimidazole methanol solution to the second sample to undergo a reaction to obtain a third sample;
(4) adding second zinc nitrate methanol solution and fourth 2-methylimidazole methanol solution to the third sample to undergo a reaction, followed by centrifuging to obtain precipitate; and
(5) dispersing the precipitate in the step (4) in water, adding aqueous solution of ethylene glycol and aqueous solution of triethanolamine to the water added with the precipitate to obtain a mixture, and stirring the mixture to obtain the ZIF-67@ZIF-8@ZIF-67@ZIF-8 hollow multilayer MOF material.

2. The preparation method of the ZIF-67@ZIF-8@ZIF-67@ZIF-8 hollow multilayer MOF material as claimed in claim 1, wherein conditions of the reaction in each of the step (1) to the step (4) comprise: stirring at a temperature in a range of 50-60 degrees Celsius (° C.) with a speed in a range of 200-600 revolutions per minute (rpm) for 5 minutes (min), followed by standing for 1 hour (h).

3. The preparation method of the ZIF-67@ZIF-8@ZIF-67@ZIF-8 hollow multilayer MOF material as claimed in claim 1, wherein a concentration of each of the first cobalt nitrate methanol solution and the second cobalt nitrate methanol solution is in a range of 0.01-0.5 mole per liter (mol/L), a concentration of each of the first 2-methylimidazole methanol solution to the fourth 2-methylimidazole methanol solution is in a range of 1-5 mol/L, a concentration of each of the first zinc nitrate methanol solution and the second zinc nitrate methanol solution is in a range of 0.01-0.5 mol/L; and a volume ratio of the first cobalt nitrate methanol solution to the first 2-methylimidazole methanol solution is in a range of 1:1-20, a volume ratio of the first zinc nitrate methanol solution to the second 2-methylimidazole methanol solution is in a range of 1:1-20, a volume ratio of the second cobalt nitrate methanol solution to the third 2-methylimidazole methanol solution is in a range of 1:1-20, and a volume ratio of the second zinc nitrate methanol solution to the fourth 2-methylimidazole methanol solution is in a range of 1:1-20.

4. The preparation method of the ZIF-67@ZIF-8@ZIF-67@ZIF-8 hollow multilayer MOF material as claimed in claim 1, wherein the centrifuging comprises: centrifuging the third sample added with the third product at a rotate speed in a range of 5000-10000 rpm for 15-30 min.

5. The preparation method of the ZIF-67@ZIF-8@ZIF-67@ZIF-8 hollow multilayer MOF material as claimed in claim 1, wherein the stirring comprises: stirring the mixture at a temperature in a range of 0-15° C. with a stirring speed in a range of 500-1000 rpm for 2-6 h.

6. The preparation method of the ZIF-67@ZIF-8@ZIF-67@ZIF-8 hollow multilayer MOF material as claimed in claim 1, wherein a concentration of the aqueous solution of the ethylene glycol is in a range of 0.2-1.2 grams per milliliter (g/mL), a concentration of the aqueous solution of the triethanolamine is in a range of 0.02-0.5 g/mL, and a ratio of the precipitate to the aqueous solution of the ethylene glycol to the aqueous solution of the triethanolamine is in a range of 1 milligram (mg):(5-10 mL):(0.5-2 mL).

7. The ZIF-67@ZIF-8@ZIF-67@ZIF-8 hollow multilayer MOF material prepared according to the preparation method in claim 1.

8. An application method for the ZIF-67@ZIF-8@ZIF-67@ZIF-8 hollow multilayer MOF material as claimed in claim 7, comprising: applying the ZIF-67@ZIF-8@ZIF-67@ZIF-8 hollow multilayer MOF material as a thrombosis drug nanocarrier to prepare drugs for thrombosis treatment.

9. An antithrombotic pharmaceutical formulation, comprising the ZIF-67@ZIF-8@ZIF-67@ZIF-8 hollow multilayer MOF material as claimed in claim 7 and an active ingredient.

10. The antithrombotic pharmaceutical formulation as claimed in claim 9, wherein the active ingredient comprises at least one selected from the group consisting of streptokinase, urokinase, plasmin, heparin sodium, rivaroxaban, and warfarin.
